(19) United States
(12) Patent Application Publication
(10) Pub. No.: US 2025/0027850 A1
(43) Pub. Date: Jan. 23, 2025

(54) GAS DETECTION EQUIPMENT

(71) Applicant: Weltall Technology Corp., Hsinchu City (TW)

(72) Inventor: Yi-Nan Kuo, Hsinchu City (TW)

(21) Appl. No.: 18/776,547

(22) Filed: Jul. 18, 2024

(30) Foreign Application Priority Data
Jul. 20, 2023 (TW) 112127205

(51) Int. Cl. G01N 1/26; G01N 1/00

(52) U.S. Cl. CPC G01N 1/26; G01N 2001/002

(57) ABSTRACT

A gas detection equipment is provided, wherein the gas detection equipment includes: a main body; and a plurality of sampling assemblies detachably connected to the main body, each of the plurality of sampling assemblies including a plurality of sampling inlets, a solenoid valve assembly and a sampling outlet, the plurality of sampling inlets being configured to be in communication with a plurality of detected gas sources, respectively, the solenoid valve assembly being connected between the plurality of sampling inlets and the sampling outlet, the solenoid valve assembly being configured to selectively control at least one of the plurality of sampling inlets to be communicated with or discommunicated from the sampling outlet.

### BACKGROUND OF THE INVENTION

### Field of the Invention

The present invention relates to a detection equipment, particularly to a gas detection equipment.

### Description of the Prior Art

In the semiconductor manufacturing process, the air quality in the cleanroom environment has a significant influence on the yield of semiconductors. However, since the factory area is extremely large, it requires a lot of manpower and time to detect the air quality by using machines or moving machines to various areas of the factory. Additionally, conventional fixed-point gas detection equipment has detection modules fixedly installed thereon. If a detection module malfunctions and needs maintenance, the entire equipment has to stop detecting the environmental gases until the maintenance is completed, resulting in low detection efficiency.

The present invention is, therefore, arisen to obviate or at least mitigate the above-mentioned disadvantages.

### SUMMARY OF THE INVENTION

The main object of the present invention is to provide a gas detection equipment which facilitates maintenance and replacement of each sampling assembly and enables multi-point detection.

To achieve the above and other objects, a gas detection equipment is provided, wherein the gas detection equipment includes: a main body; and a plurality of sampling assemblies detachably connected to the main body, each of the plurality of sampling assemblies including a plurality of sampling inlets, a solenoid valve assembly and a sampling outlet, the plurality of sampling inlets being configured to be in communication with a plurality of detected gas sources, respectively, the solenoid valve assembly being connected between the plurality of sampling inlets and the sampling outlet, the solenoid valve assembly being configured to selectively control at least one of the plurality of sampling inlets to be communicated with or discommunicated from the sampling outlet.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 is a perspective view of an exemplary embodiment of the present invention;

FIG. 2 is an enlarged perspective view of FIG. 1;

FIG. 3 is a schematic view of a second limitation unit in a locked position according to an exemplary embodiment of the present invention;

FIG. 4 is a schematic view of the second limitation unit in a release position according to an exemplary embodiment of the present invention;

FIG. 5 is a schematic view of a sampling assembly detached from a main body according to an exemplary embodiment of the present invention;

FIG. 6 is another perspective view of an exemplary embodiment of the present invention;

FIG. 7 is another perspective view of an exemplary embodiment of the present invention; and

FIG. 8 is an exploded view of the sampling assembly and a rail assembly detached from each other according to an exemplary embodiment of the present invention.

### DETAILED DESCRIPTION OF THE PREFERRED EMBODIMENTS

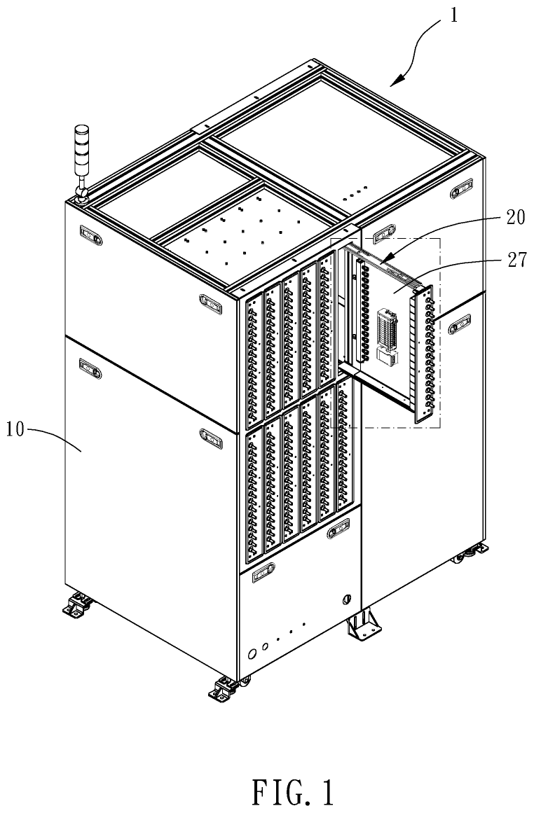

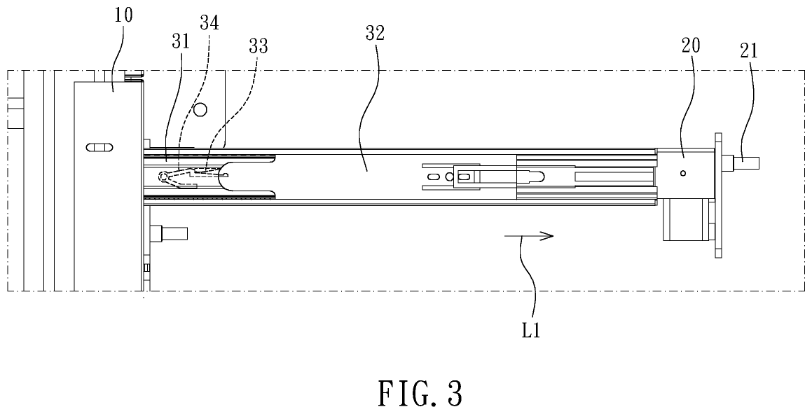

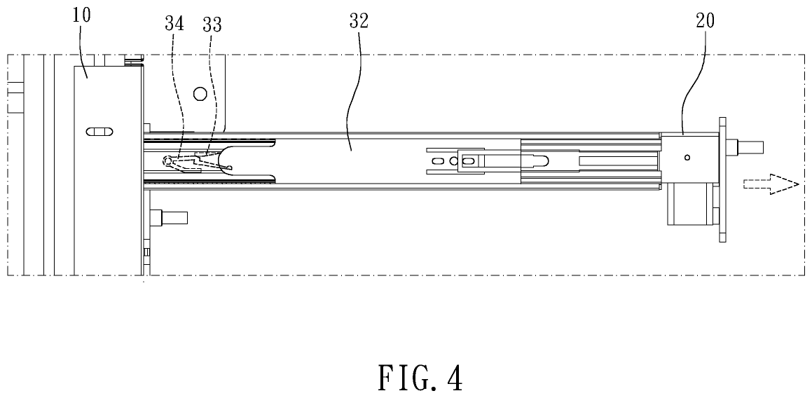

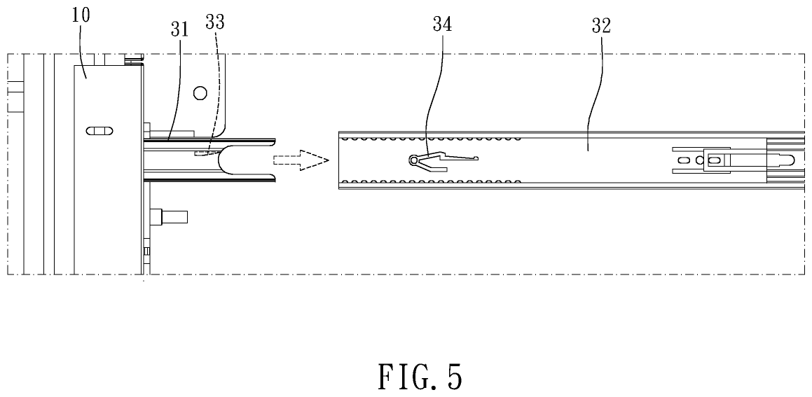

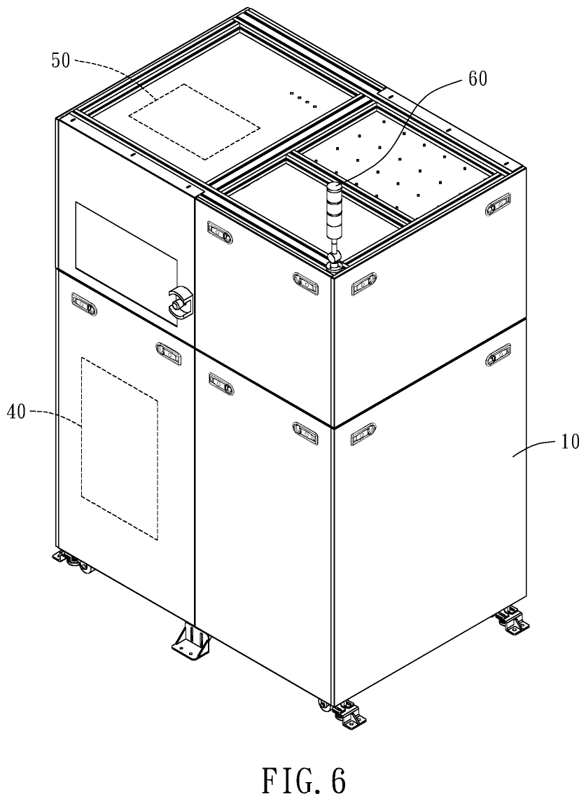

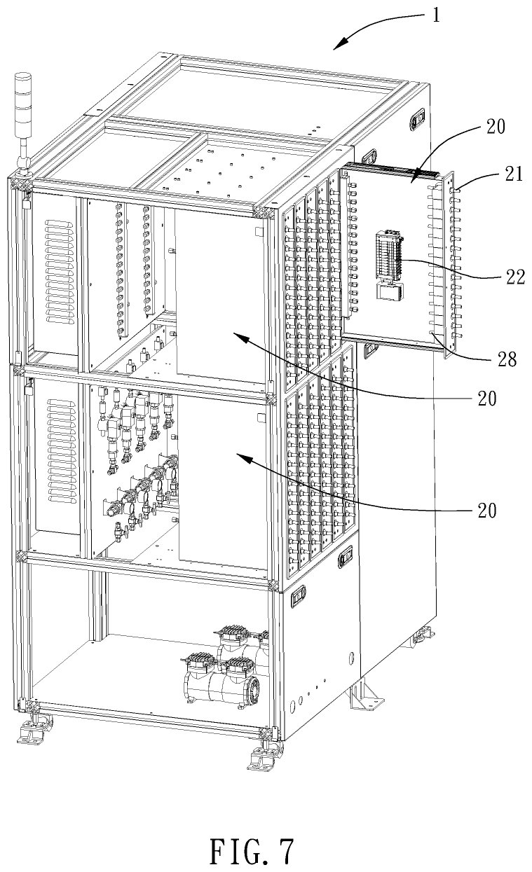

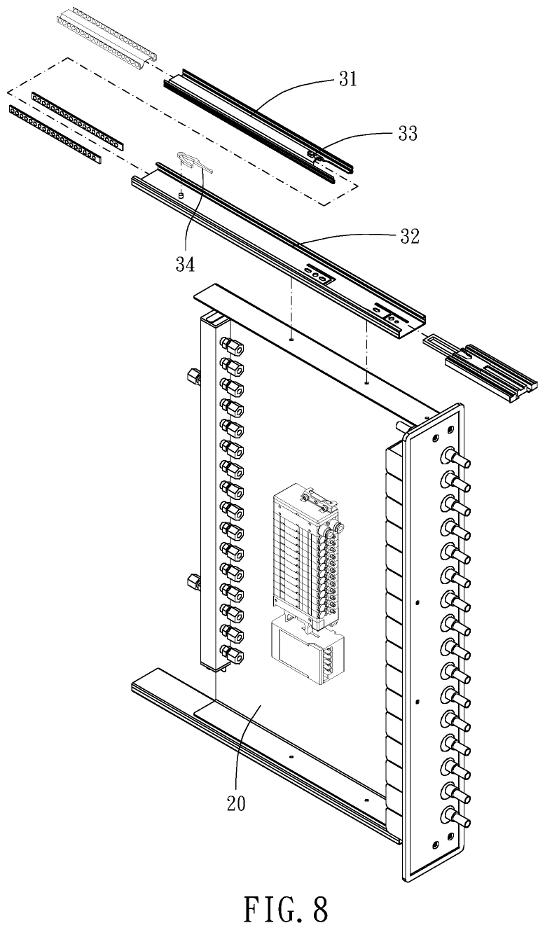

Please refer to FIGS. 1 to 8 for an exemplary embodiment of the present invention. A gas detection equipment of the present invention includes a main body 10 and a plurality of sampling assemblies 20.

The plurality of sampling assemblies 20 are detachably connected to the main body 10. Each of the plurality of sampling assemblies 20 includes a plurality of sampling inlets 21, a solenoid valve assembly 22 and a sampling outlet 23. The plurality of sampling inlets 21 are configured to be in communication with a plurality of detected gas sources, respectively. In this embodiment, the detected gas sources are the environmental gases in different work areas. The solenoid valve assembly 22 is connected between the plurality of sampling inlets 21 and the sampling outlet 23. The solenoid valve assembly 22 is configured to selectively control at least one of the plurality of sampling inlets 21 to be communicated with or discommunicated from the sampling outlet 23. This arrangement allows for independent maintenance and replacement of the plurality of sampling assemblies 20, enhancing the overall equipment's maintainability and efficiency, and enabling multi-point environmental detection across different areas with high detection efficiency. Additionally, the solenoid valve assembly 22 can quickly switch the gas to be detected from different work areas into the sampling outlet 23 for subsequent detection.

Each of the plurality of sampling assemblies 20 is detachably and slidably disposed on the main body 10. Specifically, the gas detection equipment further includes at least one rail assembly 30 arranged between the main body 10 and at least one of the plurality of sampling assemblies 20. Each of the at least one rail assembly 30 includes a first rail 31 and a second rail 32, the main body 10 includes the first rail 31, and at least one of the plurality of sampling assemblies 20 includes the second rail 32 which is slidably connected to the first rail 31. In this embodiment, the main body 10 includes a plurality of rail assemblies 30 each arranged between one of the plurality of sampling assemblies 20 and the main body 10. This facilitates the disassembly and assembly of the plurality of sampling assemblies 20, saving time and enabling smooth sliding relative to the main body 10. Furthermore, a ball structure may be arranged between the first rail and the second rail to facilitate smooth movement.

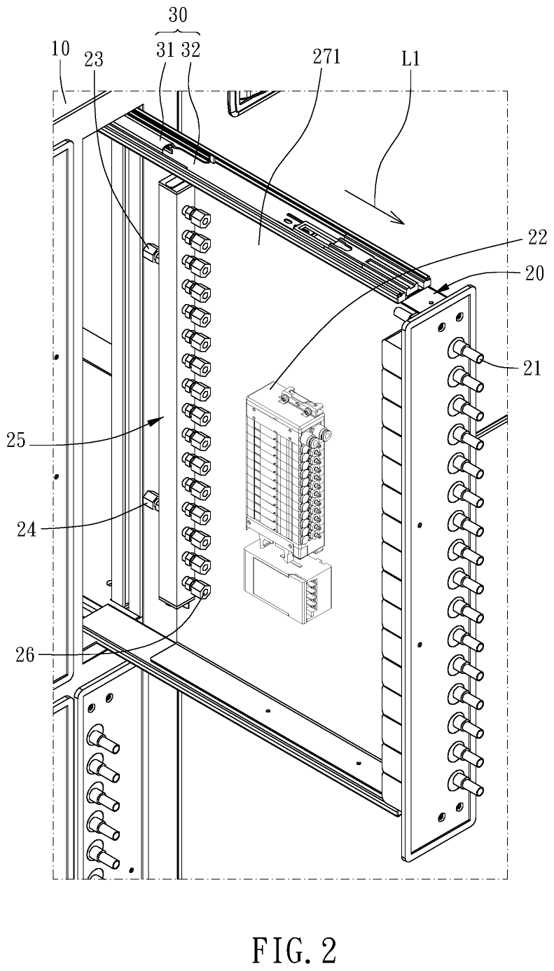

The first rail 31 includes a first limitation unit 33, and the second rail 32 includes a second limitation unit 34. Each of the plurality of sampling assemblies 20 is movable relative to the main body 10 in a first direction L1 from a retracted position to a withdrawn position outside the main body 10 (as shown in FIG. 2). When at least one of the plurality of sampling assemblies 20 is in the withdrawn position, the second limitation unit 34 abuts against the first limitation unit 33 in the first direction L1. When each of the plurality of sampling assemblies 20 is in the retracted position, they are retracted within the main body 10. Specifically, the second limitation unit 34 is movable between a locked position and a release position. When the second limitation unit 34 is in the locked position, the second limitation unit 34 abuts against the first limitation unit 33 in the first direction L1. When the second limitation unit 34 is in the release position, the second limitation unit 34 does not abut the first limitation unit 33. In this embodiment, the second limitation unit 34 is an elastic member such that each of the plurality of sampling assemblies 20 can move smoothly relative to the main body 10 in the first direction L1, simplifying disassembly and replacement operations.

The plurality of sampling inlets 21 are protrusive out beyond the main body 10, and the sampling outlet 23 is located within the main body 10, making it easier and more convenient to connect the plurality of gas detected sources while also protecting the sampling outlet 23 from damage or contamination.

Each of the plurality of sampling assemblies 20 further includes a cleaning inlet 24, the cleaning inlet 24 is in communication with the solenoid valve assembly 22, and the cleaning inlet 24 is in communication with a cleaning gas source (such as clean air or inert gases such as nitrogen). Specifically, each of the plurality of sampling assemblies 20 further includes a dispensing unit 25, the solenoid valve assembly 22 is arranged between the plurality of sampling inlets 21 and the dispensing unit 25, and the dispensing unit 25 includes a plurality of dispensing inlets 26, the sampling outlet 23 and the cleaning inlet 24. The plurality of dispensing inlets 26 are connected between the solenoid valve assembly 22 and the sampling outlet 23, and between the solenoid valve assembly 22 and the cleaning inlet 24. The cleaning inlet 24 is in communication with the cleaning gas source. This allows the plurality of sampling assemblies 20 to be cleaned, preventing different detected gases from mixing and causing detection inaccuracies, and the simple structure is conducive to installation.

Each of the plurality of sampling assemblies 20 is movable relative to the main body 10 in the first direction L1. Each of the plurality of sampling assemblies 20 further includes a plurality of connection outlets 28, and the plurality of connection outlets 28 are in communication with and between the plurality of sampling inlets 21 and the solenoid valve assembly 22. The plurality of connection outlets 28, the solenoid valve assembly 22 and the dispensing unit 25 are arranged in the first direction L1. This facilitates the connection of a plurality of pipelines between the plurality of connection outlets 28 and the solenoid valve assembly 22 and between the solenoid valve assembly 22 and the plurality of sampling outlets 23, which allows the pipelines to be neatly arranged and not interfere with each other, and enables the gas to flow smoothly within.

Each of the plurality of sampling assemblies 20 further includes a base 27, and the solenoid valve assembly 22 and the sampling outlet 23 are disposed on a first side 271 of the base 27. When each of the plurality of sampling assemblies 20 is in the withdrawn position and the first side 271 is located outside the main body 10, the solenoid valve assembly 22 and the dispensing unit 25 of each of the plurality of sampling assemblies 20 are exposed outside the main body 10. Additionally, when the first side 271 of each of the plurality of sampling assemblies 20 is inside the main body 10, the solenoid valve assembly 22 and the dispensing unit 25 of each of the plurality of sampling assemblies 20 are located within the main body 10. As such, by pulling out each of the plurality of sampling assemblies 20 without detaching it from the main body 10, the state of the solenoid valve assembly 22 and the dispensing unit 25 can be directly observed, and the pipelines between them can be directly disassembled and assembled, facilitating maintenance.

The gas detection equipment 1 further includes a detection unit 40, a processing unit 50 and an alerting device 60. The detection unit 40 and the processing unit 50 are disposed on the main body 10, the alerting device 60 is disposed on the main body 10 and connected to the processing unit 50, the detection unit 40 is in communication with the sampling outlet 23 for detecting gases from the gas detected sources, and the processing unit 50 is configured to receive detected data from the detection unit 40 for calculation and analysis. When an abnormal condition occurs, the alerting device 60 provides an alert. Each of the plurality of sampling assemblies 20 is movable relative to the processing unit 50 and the detection unit 40 in the first direction L1. In this embodiment, the alerting device 60 is a lighting device for a noticeable alert effect.

In operation, after detaching one of the sampling assemblies 20 (such as for maintenance or replacement), another sampling assembly 20 can be quickly replaced, reducing the time the gas detection is interrupted, thereby improving detection efficiency and facilitating maintenance and replacement.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A gas detection equipment including:
a main body; and
a plurality of sampling assemblies detachably connected to the main body, each of the plurality of sampling assemblies including a plurality of sampling inlets, a solenoid valve assembly and a sampling outlet, the plurality of sampling inlets being configured to be in communication with a plurality of detected gas sources, respectively, the solenoid valve assembly being connected between the plurality of sampling inlets and the sampling outlet, the solenoid valve assembly being configured to selectively control at least one of the plurality of sampling inlets to be communicated with or discommunicated from the sampling outlet.

2. The gas detection equipment of claim 1, wherein the plurality of sampling inlets of each of the plurality of sampling assemblies are protrusive out beyond the main body, and the sampling outlet of each of the plurality of sampling assemblies is located within the main body.

3. The gas detection equipment of claim 1, wherein each of the plurality of sampling assemblies further includes a cleaning inlet, the cleaning inlet is in communication with the solenoid valve assembly, and the cleaning inlet is in communication with a cleaning gas source.

4. The gas detection equipment of claim 1, wherein each of the plurality of sampling assemblies is detachably and slidably disposed on the main body.

5. The gas detection equipment of claim 1, wherein each of the plurality of sampling assemblies further includes a dispensing unit, the solenoid valve assembly is arranged between the plurality of sampling inlets and the dispensing unit, the dispensing unit includes a plurality of dispensing inlets, the sampling outlet and a cleaning inlet, the plurality of dispensing inlets are connected between the solenoid valve assembly and the sampling outlet and between the solenoid valve assembly and the cleaning inlet, and the cleaning inlet is in communication with a cleaning gas source.

6. The gas detection equipment of claim 5, wherein each of the plurality of sampling assemblies further includes a base, the solenoid valve assembly and the dispensing unit are disposed on a first side of the base, when each of the plurality of sampling assemblies is in an withdrawn position and the first side is located outside the main body, the solenoid valve assembly and the dispensing unit of each of the plurality of sampling assemblies are exposed outside the main body.

7. The gas detection equipment of claim 1, further including at least one rail assembly, wherein the at least one rail assembly is arranged between the main body and at least one of the plurality of sampling assemblies, each of the at least one rail assembly includes a first rail and a second rail, the main body includes the first rail, and at least one of the plurality of sampling assemblies includes the second rail which is slidably connected to the first rail.

8. The gas detection equipment of claim 7, wherein the first rail includes a first limitation unit, the second rail includes a second limitation unit, each of the plurality of sampling assemblies is movable relative to the main body in a first direction from a retracted position to a withdrawn position outside the main body, and when at least one of the plurality of sampling assemblies is in the withdrawn position, the second limitation unit is abutted against the first limitation unit in the first direction.

9. The gas detection equipment of claim 8, wherein the second limitation unit is movable between a locked position and a release position, when the second limitation unit is in the locked position, the second limitation unit is abutted against the first limitation unit in the first direction, and when the second limitation unit is in the release position, the second limitation unit does not abut the first limitation unit in the first direction.

10. The gas detection equipment of claim 6, wherein each of the plurality of sampling assemblies is movable relative to the main body in a first direction; each of the plurality of sampling assemblies further including a plurality of connection outlets, the plurality of connection outlets are in communication with and between the plurality of sampling inlets and the solenoid valve assembly, and the plurality of connection outlets, the solenoid valve assembly and the dispensing unit are arranged in the first direction; the plurality of sampling inlets are protrusive out beyond the main body, and the sampling outlet is located within the main body; each of the plurality of sampling assemblies is detachably and slidably disposed on the main body; when the first side of each of the plurality of sampling assemblies is inside the main body, the solenoid valve assembly and the dispensing unit of each of the plurality of sampling assemblies are located within the main body; the gas detection equipment further includes at least one rail assembly, the at least one rail assembly is arranged between the main body and at least one of the plurality of sampling assemblies, each of the at least one rail assembly includes a first rail and a second rail, the main body includes the first rail, and at least one of the plurality of sampling assemblies includes the second rail and is slidably connected to the first rail; the first rail includes a first limitation unit, the second rail includes a second limitation unit, each of the plurality of sampling assemblies is movable relative to the main body in the first direction from a retracted position to a withdrawn position outside the main body; when at least one of the plurality of sampling assemblies is in the withdrawn position, the second limitation unit is abutted against the first limitation unit in the first direction; the second limitation unit is movable between a locked position and a release position, when the second limitation unit is in the locked position, the second limitation unit is abutted against the first limitation unit in the first direction; when the second limitation unit is in the release position, the second limitation unit does not abut the first limitation unit in the first direction; the gas detection equipment further includes a detection unit, a processing unit and an alerting device, the detection unit and the processing unit are disposed on the main body, the alerting device is disposed on the main body and connected to the processing unit, the detection unit is in communication with the sampling outlet for detecting gases from the detected gas sources, and the processing unit is configured to receive detected data from the detection unit for calculation and analysis; when an abnormal condition occurs, the alerting device provides an alert; each of the plurality of sampling assemblies is movable relative to the processing unit and the detection unit in the first direction.
